(19) United States
(12) Patent Application Publication
(10) Pub. No.: US 2009/0304813 A1
(43) Pub. Date: Dec. 10, 2009

(54) VIRUCIDAL COMPOSITIONS AND USES

(76) Inventor: Stephen Spaulding Hickok, London (GB)

Correspondence Address: Workman Nydegger;1000 Eagle Gate Tower, 60 East South Temple, Salt Lake City, UT, 84111, US

(21) Appl. No.: 12/278,383

(22) PCT Filed: Feb. 6, 2007
(86) PCT No.: PCT/GB07/00394

§ 371 Date: Dec. 15, 2008

(30) Foreign Application Priority Data
Feb. 6, 2006 (GB) 0602325.3

(51) Int. Cl. A61K 33/34; A61K 31/215; A61K 33/30; A61K 33/32; A61K 33/04; A61P 31/12

(52) U.S. Cl. 424/630; 514/529; 424/641; 424/639; 424/702

(57) ABSTRACT

A composition for use in treating or preventing a respiratory virus which comprises: (a) at least one water soluble zinc, copper, selenium and/or manganese compound able in aqueous solution to dissociate into zinc, copper selenium and/or manganese ions; (b) at least one water soluble ammonium agent able in aqueous solution to dissociate into ammonium ions (c) at least one acid, and (d) water.

This invention is concerned with virucidal compositions and their use in preventing and/or treating a spectrum of viral species, but in particular for preventing and/or treating viruses which cause human or animal respiratory disease. The terms ‘treating’ and ‘treatment’ are used herein to embrace killing of the virus or viral particulates or controlling/preventing viral replication. More specifically, the invention in all its aspects is concerned with compositions useful in the treatment and/or prevention of the common cold and/or influenza and/or pneumonia-causing viruses. Even more specifically, the invention in all its aspects is concerned with compositions useful in the treatment and/or prevention of influenza viral strains including for example influenza virus of H1N1, H7N7, H9N2 or H5N1 strain, the latter responsible for avian flu associated with a high mortality rate. Infection by H5N1 strain of influenza virus, for example, is known adversely and seriously to affect the lungs as described hereafter.

There is potential for avian flu (or other H5N1 viral strains) to spread beyond infected areas. Whilst indications were that viral transmission was restricted from infected bird to human, there are concerns in view of more recent limited evidence for human to human transmission through mutation of the virus into a form transmissible between animal species.

Since avian ‘flu’ is associated with high mortality, in the event of mutation to a highly infective, inter-human transmissible form this raises the spectre of ‘pandemic’ outcomes with substantial fatalities. Whilst it remains highly desirable to treat, and where possible prevent, respiratory viral infections, it is especially desirable to provide formulations effective to treat and/or prevent the more serious respiratory viral infections including the most serious of such infections arising from respiratory viral H5N1 strains.

Unlike bacterial infections, many of which can be treated with known conventional antibacterial drugs such as antibiotics, respiratory viral infections e.g. the common cold or influenza cannot be treated with antibiotics. In the majority of respiratory viral infections, most of the presently available antiviral drugs are ineffective, and the body's own immune defence system has to fight the viral infection. Whilst this is typically the case with the common cold and influenza, it is especially so with influenza virus H5N1 strains and with influenza viral strains H1N1 and H7N7.

There is, in the relevant medical literature, considerable knowledge as to the mode of action of H5N1 viral infection and the weakness of the body's natural immune defence system to combat the virus. There are, however, very limited treatments available. One antiviral drug which may offer prophylactic and/or post-infection treatment of avian flu is oseltamivir phosphate, marketed by Roche under the Trade Name TAMIFLU. However, supplies are limited; it is significantly expensive and has potential side effects. In addition, there are concerns that resistance to Tamiflu™ might occur during a pandemic when large amounts of Tamiflu™ are likely to be taken in the many different countries that have stockpiled the drug. Active metabolites of Tamiflu™ are very stable and their accumulation in water sources may rapidly lead to influenza virus resistance.

Although vaccination is available against certain limited strains of influenza virus as a prophylactic measure, there is currently no marketed vaccine effective as a prophylactic measure against human infection by e.g. H5N1 strains of influenza virus. H5N1 viruses induce a pro-inflammatory ‘cytokine storm’ to which they are highly resistant and which severely damages the lungs and compromises immune function for combating viral infection. These factors alone make H5N1 viral strains especially problematic to prevent or treat with most of the available antiviral drugs.

We have now surprisingly found that a specific narrow selection of mineral—or other metallic element containing compositions disclosed as a broad range of multi-functional products in our earlier patent publication WO 01/15554, and combinations thereof, are unexpectedly useful in the treatment of respiratory viral infections, including prophylactic treatments against infection by H5N1 viral strains. We have also surprisingly found that these selected compositions, and particular combinations of such metallo-ions, can be blended into novel gel and other carrier formulations and used in sprays or hand wipe substrates so as to provide for example the following embodiments of the present invention, namely:
(i) nasal sprays and gel barrier formulations useful as prophylactic treatment to assist in prevention of infection by respiratory virus, especially influenza virus of H5N1 strain; in preferred embodiments the viscosity is less than 4750 centipoise, such as less than 4500 centipoise, preferably less than 4000 centipoise, ideally less than 3000 centipoise, such as in the range of 1 to 2500 centipoise.
(ii) cleansing gels which exhibit powerful virucidal properties useful to control (i.e. to kill, render harmless or otherwise interfere with replication of) live virus or viral particles, and
(iii) hand wipe substrates, such as disposable wipes impregnated with the compositions, and optionally modified by thickening agents or stabilisers.

We also provide embodiments in suitable oral dosage form, for example, iv) oral dosage formulations based on liquid aqueous compositions. The aqueous compositions can have single or multiple metallo-ionic species selected from a particular range. The aqueous compositions may entirely consist of the active components or may essentially consist of the said active components. In other embodiments, the metallo-ionic compositions may include optional additives such as one or more excipients and/or diluents.

Accordingly, in one aspect the present invention provides for use in treating a respiratory virus a composition comprising:
(a) at least one water soluble zinc, copper, selenium and/or manganese compound able in aqueous solution to dissociate into zinc, copper, selenium and/or manganese ions;
(b) at least one water soluble ammonium agent able in aqueous solution to dissociate into ammonium ions
(c) at least one acid, and
(d) water
   said composition preferably having a pH of less than 4 and preferably an electrolytic potential in excess of 50 milivolts.

In another aspect the invention provides a composition as defined above for use in treating an influenza virus, such as influenza virus of H1N1, H7N7, H9N2 or H5N1 strain, preferably of H5N1 strain.

In yet another aspect the invention provides a composition as defined above for use in treating viruses responsible for causing common cold symptoms.

In a still further aspect, the present invention provides compositions as defined above for use in the preparation of a medicament for use in treating respiratory viruses such as influenza viruses, preferably influenza virus of H5N1 strain, alternatively common cold viruses or a pneumonia virus. 60% of common colds are caused by rhinoviruses. The rest occur due to infection by coronavirus, influenza viruses, parainfluenza virus, respiratory syncytial virus, adenovirus, and enterovirus. In addition, the bacteria Mycoplasma pneumoniae and Chlamydia pneumoniae may cause common cold symptoms.

The compositions defined above may further include one or more additives selected from vitamin B1, B, B5, B6, C, malic acid, a natural diuretic, melatonin and valerian. The composition may essentially or preferably entirely consist of the stated components. Most preferably the water is distilled water.

The compound of zinc, copper, selenium and/or manganese is preferably an inorganic salt thereof such as the phosphate, sulphate, nitrate or chloride.

The ammonium agent, capable of binding, complexing or otherwise sequestering the zinc, copper and/or selenium ions is conveniently an inorganic ammonium salt or ammonium hydroxide capable of dissociating in aqueous solution into ammonium ions. For example, one or more of the following may be present in the composition: ammonium hydroxide, sulphate, chloride, phosphate, nitrate, citrate and tartarate.

The at least one acid is conveniently selected from the following group: sulphuric, hydrochloric, nitric, phosphoric, citric and tartaric acids. The at least one acid can be for example concentrated sulphuric, hydrochloric, nitric or phosphoric acids.

The pH of the composition can be less than 4, such as 3.5 or less, 3 or less, 2.5 or less such as 2 or less such as in the range of 1 or less. The electrolytic potential can be for example, in excess of 100 mV, or in excess of 150 mV, or in excess of 200 mV, or in excess of 250 mV, or in excess of 300 mV or in excess of 340 mV such as in the range up to 400 mV.

The compositions encompassed by the present invention can be made as outlined in the ‘general procedure’ section in our earlier patent publication and as exemplified therein, where the metallic or other mineral element in the table of examples therein is at least one of copper, zinc, selenium and manganese. When using compositions with more than one metallo-ionic species, copper and zinc may be present with or without selenium; Selenium and zinc may be used together, with or without copper. Copper and selenium may be present together, with or without zinc. All may be present with manganese or it may be used alone. A suitable manganese preparation can be made by extrapolating from the general procedure outlined above, using e.g. manganese sulphate, sulphuric acid and ammonium sulphate to arrive at a composition pH of 2 or less and a potential in excess of 250 milivolts.

The invention further provides antiviral compositions as defined above having antiviral activity against respiratory infections including seasonal influenza, H5N1 or avian influenza, acute coryza. Such compositions may include one or more conventional pharmaceutically acceptable carriers, diluents and/or or excipients, such as saline as a diluent.

In order to illustrate the present invention the following purely non-limiting examples, presented in our earlier patent publication WO 01/015554 were used to make suitable compositions. They were prepared according to the same general procedure outlined in our earlier said patent publication. In Table 1 below, the following examples according to the present invention correspond with the stated numbered prior examples thus.

TABLE 1

| Example No. | Example No. | |
|---|---|---|
| (present invention) | (earlier publication) | Ionic species present |
| 1 | 1 | Cu²⁺, (NH₄)⁺ |
| 2 | 2 | Cu²⁺, (NH₄)⁺ |
| 3 | 3 | Cu²⁺, (NH₄)⁺ |
| 4 | 4 | Cu²⁺, (NH₄)⁺ |
| 5 | 11 | Se²⁺, (NH₄)⁺ |
| 6 | 12 | Se²⁺, (NH₄)⁺ |
| 7 | 15 | Zn²⁺, (NH₄)⁺ |
| 8 | 16 | Zn²⁺, (NH₄)⁺ |
| 9 | 17 | Zn²⁺, (NH₄)⁺ |
| 10 | 18 | Zn²⁺, (NH₄)⁺ |
| 11 | 18a | Zn²⁺, (NH₄)⁺ |
| 12 | 19 | Cu²⁺, (NH₄)⁺ |
| 13 | 20 | Cu²⁺, (NH₄)⁺ |
| 14 | 21 | Cu²⁺, (NH₄)⁺ |
| 15 | 22 | Cu²⁺, (NH₄)⁺ |
| 16 | 23 | Zn²⁺, (NH₄)⁺ |
| 17 | 24 | Cu²⁺, (NH₄)⁺ |
| 18 | 25 | Cu²⁺, (NH₄)⁺ |
| 19 | 26 | Cu²⁺, (NH₄)⁺ |
| 20 | 27 | Cu²⁺, (NH₄)⁺ |
| 21 | 29 | Cu²⁺, (NH₄)⁺ |
| 22 | 30 | Cu²⁺, (NH₄)⁺ |
| 23 | 31 | Cu²⁺, (NH₄)⁺ |
| 24 | 32 | Cu²⁺, (NH₄)⁺ |
| 25 | 33 | Cu²⁺, (NH₄)⁺ |
| 26 | 34 | Cu²⁺, (NH₄)⁺ |
| 27 | 35 | Cu²⁺, (NH₄)⁺ |
| 28 | 36 | Cu²⁺, (NH₄)⁺ |
| 29 | 37 | Cu²⁺, (NH₄)⁺ |
| 30 | 38 | Cu²⁺, (NH₄)⁺ |
| 31 | 43 | Zn²⁺, (NH₄)⁺ |
| 32 | 45 | Zn²⁺, (NH₄)⁺ |

Compositions as defined and exemplified above may be used in at least 3 treatment regimes to combat respiratory, especially influenza and more particularly H5N1 influenza viruses. They may be used as a prophylactic treatment to block entry of the virus into the nasal mucous membranes [typically the most common site of entry] and/or to kill the virus or viral particulates. Such blockage may be effected by application of nasal gels or aqueous sprays, i.e. a pharmaceutically acceptable gel or spray carrier medium containing at least one composition as defined and/or exemplified above. The virus, where present, may also be controlled by such anti-infective gel formulations as hand cleansing gels, i.e. the composition can be formulated into a topically acceptable gel medium known per se, but providing by the admixture of two known entities a novel antiviral gel or spray formulation. Furthermore, hand wipe substrates such as disposable tissues may be impregnated or coated with the compositions optionally modified to improve adherence. It is preferred to use at least the copper containing compositions in nasal gels, cleaning gels and hand wipes as a means for blocking viral entry and/or killing or controlling the virus or viral particulates.

In order to demonstrate activity of the present compositions against respiratory viruses, some were prepared and tested against H5N1 influenza viruses in vitro and in vivo to show potential for managing avian flu. The anti-oxidant properties of selenium based compositions is indicative of direct potential for virucidal activity. Three principal embodiments are presented based on single zinc, copper and selenium based compositions, and a fourth embodiment directed to the use sequentially of the present manganese, zinc and selenium metallo-ion compositions namely:

1) Virucidal activity against human H5N1 virus in vitro murine studies (Zn, Cu & Se)
2) Toxicology and dose ranging study in mice (Zn),
3) Prophylactic activity in mice infected with a lethal H5N1 influenza virus (Zn), and
4) Human case study where conventional influenza viral infection is attenuated by a combination therapy involving sequential internal administration of doses of manganese, zinc and selenium compositions.

### EXAMPLE 1

### Virucidal Activity Against Human H5N1 In Vitro

In the in vitro virucidal assay, the A/Vietnam/1203/04 (H5N1)×Ann Arbor/6/60 influenza virus was exposed to the copper metallo-ion compositions (equivalent to elemental copper at 3 ppm), the zinc metallo-ion compositions (equivalent to elemental zinc at 2 ppm) and the selenium metallo-ion compositions (equivalent to elemental selenium at 1 ppm) or to water (control) for 10 minutes at room temperature. The treated viruses were then assessed for infectivity using the standard MDCK cell assay.

The results in Table 2 below show that while the zinc and selenium compositions at their exceptionally low concentrations showed no significant direct antiviral effect, the copper compositions caused a >90% reduction in virus titre despite a similarly exceptionally low concentration.

TABLE 2

| | Concentration (ppm) | |
|---|---|---|
| | Initial low dosages in | Log 10 Reduction |
| Sample | view of acidity/pH | in virus Titre |
| Cu | 3 | 1.75 +/− 0.00 ** |
| Zn | 2 | 0.25 +/− 0.35 |
| Se | 1 | 0.88 +/− 0.88 |
| Water | N/A | 0.00 +/− 0.00 |

** statistically different from water (p < 0.001)

These results suggest that a nasal gel containing at least 3 ppm (=3 μg/ml) of the ionically modified copper compositions could provide not only a barrier to viral entry into the upper respiratory tract but also may kill a significant percentage of the viral particles encountering the gel. Since influenza viruses can be picked up from infected surfaces and transferred to the nose and eyes where entry occurs, use of hand wipes or hand gels containing at least 3 ppm of the copper compositions as a prophylactic measure can be of substantial benefit for reducing or preventing infection especially during a pandemic.

TABLE 3
Effect of Oral Treatment with ZnAL42 on an Influenza A (H5N1) Virus Infection in Mice.
Animals: Female 18-21 g BALB/c mice
Virus: Influenza A/Duck/MN/1525/81 (H5N1)
Drug diluent: Saline
Treatment schedule: as described below
Treatment route: p.o.
Expt. duration: 21 days

| | | | Tox Controls | | | | |
|---|---|---|---|---|---|---|---|
| | | | | Mean Host | | Infected, Treated Mice | |
| | Dosage | | Surv/ | Weight | Surv/ | Mean Day to | Mean Day 11 |
| Treatment | (mg/kg/day) | Treatment Schedule | Total | Change^{a} (g) | Total | Death^{b} ± SD | SaO₂ (% ± SD) |
| ZnAL42 | 17.28 | qd × 7 beg −48 h | 3/3 | −0.7 | 1/10 | 9.2 ± 3.0 | 75.3 ± 0.7 |
| | 8.74 | qd × 7 beg −48 h | 3/3 | 1.1 | 3/10 | 8.4 ± 1.1 | 76.7 ± 2.8* |
| | 17.28 | bid × 7 beg −48 h | 3/3 | 2.7 | 8/10*** | 7.0 ± 1.4 | 81.0 ± 3.5*** |
| | 8.74 | bid × 7 beg −48 h | 3/3 | 0.3 | 5/10* | 10.8 ± 3.5** | 78.7 ± 2.6** |
| | 17.28 | tid × 7 beg −48 h | 3/3 | 0.5 | 4/10 | 9.5 ± 1.6** | 76.6 ± 2.0* |
| | 8.74 | tid × 7 beg −48 h | 3/3 | 1.9 | 3/10 | 8.6 ± 2.2 | 76.2 ± 1.8 |
| | 17.28 | qid × 7 beg −48 h | 3/3 | −0.9 | 2/10 | 9.4 ± 2.5* | 76.0 ± 2.0 |
| | 8.74 | qid × 7 beg −48 h | 3/3 | −0.5 | 2/10 | 7.9 ± 1.1 | 75.4 ± 0.8 |
| Ribavirin | 75 | bid × 5 beg −4 h | 3/3 | −0.8 | 10/10*** | >21.0 ± 0.0*** | 86.0 ± 2.0*** |
| Saline | — | qid × 7 beg −48 h | — | — | 2/20 | 9.2 ± 3.0 | 75.4 ± 0.8 |
| Normal Controls | — | — | 5/5 | 1.9 | — | — | 86.0 ± 1.2*** |

^{a}Difference between initial weight and weight 18 h after final treatment.
^{b}Mean day to death of mice dying prior to day 21.
*P < 0.05;
**P < 0.01;
***P < 0.001 compared to saline-treated controls.

### EXAMPLE 2

### Toxicology and Dose Ranging Study in Mice

A pilot in vivo toxicological/dose-ranging study was performed in mice to ensure the safety of the zinc metallo-ion compositions at dosage levels before commencing the prophylaxis study. Groups of 3 mice received the Zn formulations at (i) four times human recommended daily allowance (human RDA being 15 mg of elemental zinc per day) (ii) twelve times human RDA (equivalent to 1× mouse RDA) and (iii) thirty six times human RDA once daily, orally, for 14 days.

The mice were weighed before treatment started (and 18 hours after the final treatment) and observed for signs of toxicity during the dosing period. No signs of toxicity were noted. A general examination of internal organs was performed on the mice at the end of the dosing period and no overt abnormalities were observed.

### EXAMPLE 3

Prophylactic activity in mice infected with lethal H5N1 influenza virus; namely Influenza A/Duck/MN/1525/81 (H5N1) adapted to mice.

For the prophylaxis study, groups of 10 female BALB/c (6-8 week old, around 20 g) mice received orally the zinc metallo-ion compositions diluted in distilled water at 1× and 36× human RDA ( 1/12 and 3× mouse RDA equivalent) for 3 days divided into 2 doses i.e. 1 dose am and 1 dose pm daily. The placebo group (20 mice) received distilled water twice daily.

Four hours after the last dose, mice were inoculated intranasally with the aforesaid lethal H5N1 Virus. The dosing was continued twice daily until the end of the study (21 days or mouse death).

Another group of 10 mice received Tamiflu™ (oseltamivir phosphate) orally twice daily at 10 mg/kg for 5 days, starting four hours after intranasal virus inoculation. Blood oxygen levels were measured daily (providing an indication of lung function) and the mice were weighed at the beginning and end of the study.

Results up to day 19 are summarised in table 3A below, full results are in Table 3.

TABLE 3A

| | | | Number of surviving mice |
|---|---|---|---|
| Treatment | Dose (mg/kg) | | per group at day 19 |
| Zn | 8.74 | (3 × human RDA) | 8/10 [80%] |
| Zn | 0.24 | (1/12 human RDA) | 5/10 [50%] |

| | | |
|---|---|---|
| Water/control | Nil | 8/20 [40%] |
| Tamiflu ™ | 10 | 10/10 [100%] |

### EXAMPLE 4

### Influenza—Infected Human Case Study

Key ZnAL42≡Example 7, SePC33≡Example 5 MnAL42=manganese metallo ion formulation based on a soluble manganese compound prepared according to the procedure described above.

An asthmatic subject who suffers badly from the effects of respiratory viruses has found on four separate occasions that taking a combination of ZnAL42, MnAL42 and SePC33 at the RDA level three times daily for 3-4 days when a cold or 'flu is starting (recognized by typical cold and 'flu prodrome effects and wheezing) completely prevents the development of the cold or 'flu. Without the combination of metallo-ion formulations this individual usually requires 4-6 days of bed rest and usually develops a chest infection that further exacerbates respiratory difficulties and prolongs the course of the illness to 2 weeks.

Several other non-asthmatic individuals regularly using the ZnAL42 product find that when symptoms of a cold or 'flu infection are starting, the infection can be curtailed by increasing the ZnAL42 dose to 3-times RDA for 2 to 5 days.

It has been reported by Johnston's group that the production of interferon-beta and -lambda by rhinovirus-infected bronchial epithelial cells from asthmatics is impaired compared to non-asthmatics. Thus, it is postulated without being bound by theory that ZnAL42 or the combination of ZnAL42, MnAL42 and SePC33 enhances anti-viral interferon production thereby ameliorating the viral infection and its pathogenic effects in the lungs.

There are now provided examples of gel based compositions containing copper metallo-ion composition.

### EXAMPLE 33

### Gel Based Composition

TABLE 5

| | RAW MATERIAL USED | % by wt | |
|---|---|---|---|
| PHASE A | ALOE VERA GEL BASE | 99.00 | |
| PHASE B | CU AL 42 | 1.0 | |
| * | Copper metallo-ion Composition | | ≡314 ppm Cu |

Preparation:
Step 1. Add Phase B to Phase A mixing thoroughly.
N.B. the Aloe Vera Gel Base is preferably made by Hydrating 200:1 Aloe Barbadensis Flake thickening with 0.5% xanthan gum and when clear adding 0.1% Potassium Sorbate and 0.1% Sodium Benzoate.
* Note:
see Example 34 below.

### EXAMPLE 34

### Improved Gel Based Composition

TABLE 6

| | RAW MATERIAL USED | % by wt | |
|---|---|---|---|
| A | PURIFIED WATER | 95.8 | |
| | ALOE BARBADENSIS FLAKE 200:1 | 0.5 | |
| B | XANTHAN GUM | 0.5 | |
| C | WATER | 2.0 | |
| | POTASSIUM SORBATE | 0.1 | |
| | SODIUM BENZOATE | 0.1 | |
| D | CU AL 42 | 1.0 | |
| * | Copper metallo-ion Composition | | ≡314 ppm Cu |

Preparation:
Step 1. Slowly dissolve flake aloe in water in phase A
Step 2. Heat phase A to 38-40 C.
Step 3. Sift xanthan gum into an established vortex in the water phase. Allow 45 minutes for the xanthan to dissolve completely.
Step 4. Add predissolved Potassium Sorbate and Sodium Benzoate - phase C - to gel mixture. Mix thoroughly.
Step 5. Add phase D - CU AL 42 - mix thoroughly
* Note:
An apparent excess of copper metallo ion composition is blended into the gel formulations, compared to the aqueous formulations. Despite the high level, the aloe base appears to mask undesirable metallic aftertastes when applied, e.g. to nasal passage.

1. A composition for use in treating or preventing a respiratory virus which comprises
(a) at least one water soluble zinc, copper, selenium and/or manganese compound able in aqueous solution to dissociate into zinc, copper selenium and/or manganese ions;
(b) at least one water soluble ammonium agent able in aqueous solution to dissociate into ammonium ions
(c) at least one acid, and
(d) water.

2. A composition for use in treating or preventing a respiratory virus which essentially consist of:
(a) at least one water soluble zinc, copper, selenium and/or manganese compound able in aqueous solution to dissociate into zinc, copper selenium and/or manganese ions;
(b) at least one water soluble ammonium agent able in aqueous solution to dissociate into ammonium ions
(c) at least one acid, and
(d) water.

3. A composition for use in preparing a medication for use in treating or preventing a respiratory virus, which composition comprises:
(a) at least one water soluble zinc, copper, selenium and/or manganese compound able in aqueous solution to dissociate into zinc, copper selenium and/or manganese ions;
(b) at least one water soluble ammonium agent able in aqueous solution to dissociate into ammonium ions
(c) at least one acid, and
(d) water.

4. A composition for use in preparing a medicament for use in treating or preventing a respiratory virus, which composition essentially consists of
(a) at least one water soluble zinc, copper, selenium and/or manganese compound able in aqueous solution to dissociate into zinc, copper selenium and/or manganese ions;
(b) at least one water soluble ammonium agent able in aqueous solution to dissociate into ammonium ions
(c) at least one acid, and
(d) water.

5. A composition as claimed in any preceding claim having a pH of less than 4.

6. A composition as claimed in any preceding claim having an electrolytic potential in excess of 50 milivolts.

7. A composition as claimed in any preceding claim further including an excipient or diluent.

8. A composition as claimed in claim 7 in which the diluent comprises saline.

9. A composition as claimed in any preceding claim in the form of a virucidal gel.

10. A composition as claimed in any one of claims 1 to 8, in the form of a spray such as a spray adapted for nasal use.

11. A composition as claimed in any one of claims 1 to 8 in the form of a substrate impregnated with the composition.

12. A composition as claimed in any preceding claim, wherein the virus to be treated or prevented is an influenza virus.

13. A composition as claimed in claim 12 wherein the virus is of H1N1, H7N7, H9N2 or H5N1 strain.

14. A composition as claimed in any preceding claim, wherein the strain is H5N1 and associated with causing avian influenza.

15. A composition as claimed in any one of claims 1 to 11, wherein the virus to be treated or prevented is a respiratory virus responsible for causing common cold symptoms.

16. A composition according to any one of claims 1 to 11 in combination with another antiviral preparation.

17. A composition as claimed in claim 16 in which the other preparation is oseltamivir phosphate.

18. A composition as claimed in any preceding claim, wherein at least two different such metal-ion species are present.

19. A composition as claimed in claim 18 in which metallo-ions of zinc are present together with one or more of copper, selenium and manganese metallo ions.

20. A composition as claimed in any one of claims 9 to 19 in which the gel is based on aloe barbadensis.

21. A composition as claimed in claim 20 in which the gel comprises at least one thickener such as xanthan gum.

22. A composition as claimed in claim 21 in which at least one preservative is present.

23. A composition as claimed in any one of claims 9 or 20 to 22 in which the gel contains copper metallo ions.

24. A composition as claimed in claim 23 in which the amount of copper ions present in the gel is at the level of 100 to 500 ppm, preferably 150 to 450 ppm, more preferably 200 to 350 ppm.
